(19) United States
(12) Patent Application Publication
(10) Pub. No.: US 2017/0119818 A1
(43) Pub. Date: May 4, 2017

(54) ANTICANCER NANO-SILVER COMPOSITION FOR TREATMENT OF LUNG CANCER, AND PREPARATION METHOD AND USE THEREOF

(71) Applicant: CHANGSHA DIGU NANO BIOTECHNOLOGY CO., LTD., Changsha (CN)

(72) Inventors: Jinjun LIU, Changsha (CN); Weiyi SITU, Changsha (CN); Qiangbai LI, Changsha (CN)

(73) Assignee: CHANGSHA DIGU NANO BIOTECHNOLOGY CO., LTD., Changsha (CN)

(21) Appl. No.: 15/330,759

(22) PCT Filed: May 8, 2015
(86) PCT No.: PCT/CN2015/000315

§ 371 Date: Nov. 3, 2016

(30) Foreign Application Priority Data
May 9, 2014 (CN) 201410194184.X

(51) Int. Cl. A61K 33/38; A61K 9/14; A61K 47/26; A61K 47/32; A61K 47/18

(52) U.S. Cl. CPC A61K 33/38; A61K 47/32; A61K 47/18; A61K 47/26; A61K 9/14

(57) ABSTRACT

The present invention provided an anticancer nano-silver composition for the treatment of lung cancer. The composition contains spherical nano-silver powder 12-200 mg/kg, pharmaceutically acceptable dispersing agent carbomer 700-800 mg/kg, triethanolamine 700-800 mg/kg, glucose 2.8-3.2 g/kg, and purified water as the diluent; of the spherical nano-silver powder, the purity of silver is ≧99.99% and the particle size is 1-5 nm. Experiments demonstrated that the anticancer nano-silver composition can be used to inhibit proliferation of the human non-small cell lung cancer A549, and cause all cells death. The nano-silver composition of the present invention can be used in the manufacture of medicaments and for the treatment of lung cancer.

### FIELD OF THE INVENTION

The present invention relates to a nano-silver composition for the treatment of lung cancer, and preparation method and use thereof.

### BACKGROUND OF THE INVENTION

Lung cancer is considered as one of the most severe malignant cancers with rapidly increased morbidity and mortality, which poses a great threat to human health and life. The morbidity and mortality of lung cancer have been significantly increased in many countries as reported for the recent fifty years. Lung cancer is ranked the first among all malignant cancers in both morbidity and mortality for men, and ranked the second among all malignant cancers in both morbidity and mortality for women. Though the cause of lung cancer is still not fully clear, but a large number of data showed that the occurrence of lung cancer is closely related to long-term and heavy smoking. Based on study results, the risk of suffering lung cancer is 10 to 20 times greater for long-term and heavy smokers than non smokers. The younger a person starts smoking, the greater the risk of suffering lung cancer. Furthermore, smoking not only directly affects the health of the smokers, but also has adverse effects on the health of the people within the environment, such as causing the disease among the “passive” smokers. The morbidity of lung cancer in cities is higher than that in rural areas, which may be related to air pollutants and dusts which contain carcinogens.

Currently, lung cancer is treated mainly through chemotherapy, radiotherapy and surgery. Each type of the above treatments may cause great harms to the body but still cannot completely cure the cancer. There is a high risk of recurrence after a patient has lung cancer treatment.

Nano-silver materials are a type of new functional materials developed recently through nano-technologies. Nano-scaled metallic silver powder is prepared from elemental metallic silver or silver-containing compounds by physical or chemical methods, and the powder may have a particle size of less than 100 nm. As in the size of nano-scale, the materials have very high surface area with much exposed atomic bonds and electrons, and thus the activity is significantly increased. As a result, the metallic silver shows some novel in terms of physical and chemical properties. One of the most significant functions of nano-silver is related to the enhanced antibacterial properties. In the National Institution of High Energy Physics, the scientists for nano biological effects recently discovered that nano-particles with chemical modification demonstrated relatively high efficiency on inhibition to tumor growth without killing the cells directly, and that such nano-particles can be used to enhance the immunity in the mice with tumor and they are almost nontoxic. They are much different from traditional antitumor drugs and are really desirable for the treatment of cancers. It is interesting for the scientists that such nano-particles need not directly kill the cells, and their distribution in the tumor tissues can be very low, only about one out of a hundred thousands. Such results suggest that the inhibitory effect of the nano-particles is achieved not by direct killing of the cells and therefore there may be some other unknown anti-cancer mechanisms for further investigation. The application of nano-technology shall facilitate the discovery of new anti-cancer drugs.

Currently, most of the clinical used anti-tumor drugs are highly toxic for killing cells, while killing the tumor cells the drugs also seriously damage the normal cells. The above-mentioned studies avoided such toxicity problem, and thus were considered as of a new approach for the treatment of cancer with high efficiency and low toxicity. However, the above studies used chemically modified nano-particles, which may have drug safety concerns, and further there were no any reports regarding whether the cell killing effect is due to the chemical modification or the nano-particles themselves.

Currently, in mostly domestic manufactures, nano-silver powders are often prepared via chemical methods in varied particle sizes and shapes, and thus it would be difficult to control the purity of batch production. It is hard to find nano-silver powders with high safety and purity, and uniformity in size and shape. Nano-sliver materials with antibacterial properties may have been used in fabrics and for the treatment of inflammatory diseases of female genital tract, but there is no any reports regarding applications on tumors.

### SUMMARY OF THE INVENTION

To solve the technical problems of the prior art mentioned above, the present invention is directed to provide a nano-silver composition for the treatment of lung cancer and method for preparation thereof.

In one aspect, the present invention provided an anticancer nano-silver composition for the treatment of lung cancer, the composition, on a basis of per kilogram of total weight, comprising:

nano-silver powder 12-200 mg;

carbomer 700-800 mg;

triethanolamine 700-800 mg;

glucose 2.8-3.2 g;

water as remaining;

wherein, purity of silver of the nano-silver powder is ≧99.99%, and particles of the nano-silver powder are 1-5 nm in size and spherical in shape. The nano-silver powder is the No.5 nano-silver purchased from the Hunan Optics Valley Nano Technology Co., Ltd.

In a further aspect, the present invention provided a method for preparation of the anticancer nano-silver composition. The method includes the following steps:

(1) Preparing the nano-silver powder, carbomer, triethanolamine, glucose and water in a ratio defined in claim 1;

(2) adding the carbomer into ½ to ⅔ of total amount of the water with mixing to obtain a mixture, which is then dispersed in a ultrasonic disperser for 2-5 min;

(3) adding the nano-silver powder and the triethanolamine into the mixture dispersed in step (2) to obtain a mixture, which is then dispersed in a ultrasonic disperser for 2-5 min;

(4) adding the glucose and remaining water into the mixture dispersed in step (3) to obtain a mixture, which is then dispersed in a ultrasonic disperser for 1-3 min;

(5) cooling the mixture dispersed in step (4) to 2-12° C. to obtain a mixture, which is dispersed in an ultrasonic atomizer into a mist, which is collected in a collecting device and is condensed to form an anticancer nano-silver composition;

wherein, the ultrasonic disperser used in step (2) is of band 2 frequency: 16-24 KHz; the ultrasonic disperser used in step (3) is of band 5 frequency: 40-65 KHz; the ultrasonic disperser used in step (4) is of band 4 frequency: 30-40 KHz; the ultrasonic atomizer used in step (5) is of band 15 frequency: 120-180 KHz.

The No.5 nano-silver powder as used in the invention is of high-purity elemental silver nano-particles, and the particles are as required in spherical shape and 1-5 nm in size. The spherical shape makes the silver nano-particles to be maximal in the specific surface area and stability. If the particle size is greater than 5 nm, as the volume of the particles increases, the unit surface area and activity decrease, and the required drug dosage increases; but if the particle size is less than 1 nm, the manufacturing process would be found quite difficult and very costly. Taking account of drug safety, procedural efficiency and cost-effectiveness, the particle size of the nano-silver powder of the present invention is defined as in the range of 1 nm to 5 nm. The nano-silver composition used in the present invention is highly safe and is of high purity, high efficacy, high stability and low concentration, and it can be manufactured with auxiliary materials through a special process to deliver a drug system of highly dispersible, highly stabilized and readily releasable for drug dispersion, preservation and release.

The experiments showed that the anticancer nano-silver composition of the invention can strongly inhibit proliferation of the human non-small cell lung cancer A549, and cause cell death. It is indicated that the anticancer nano-silver composition can be used to manufacture medicaments for the treatment of lung cancer

### BRIEF DESCRIPTION OF THE DRAWINGS

FIG. 1 illustrates the effects of the Nano-Silver 5# on cell proliferation.

FIG. 2 illustrates the effects of the Nano-Silver 1# on cell proliferation.

### DETAILED DESCRIPTION OF THE EMBODIMENTS

The nano-silver powder used in this example is of the No.1 nano-silver (0 ppm) with a batch number of 2013.05.21-11 and the No.5 nano-silver (250 ppm) with a batch number of 2013.05.21-15, provided by the Hunan Optics Valley Nano Technology Co., Ltd.

### EXAMPLE 1

A nano-silver composition for the treatment of lung cancer includes on a basis of per kilogram of total weight:

nano-silver powder 0.0625 g;

carbomer 0.7000 g;

triethanolamine 0.7000 g;

glucose 3.0000 g;

water 995.5385 g;

wherein, the purity of silver of the nano-silver powder is ≧99.99%, the particles of the nano-silver powder are 1-5 nm in size and are in spherical shape.

### EXAMPLE 2

A nano-silver composition for the treatment of lung cancer includes on a basis of per kilogram of total weight:

nano-silver powder 0.0125 g;

carbomer 0.8000

g;

triethanolamine 0.8000 g;

glucose 3.0000 g;

water 995.3885 g;

wherein, the purity of silver of the nano-silver powder is ≧99.99%, particles of the nano-silver powder are 1-5 nm in size and are in spherical shape.

### EXAMPLE 3

A nano-silver composition for the treatment of lung cancer includes on a basis of per kilogram of total weight:

nano-silver powder 0.0025 g;

carbomer 0.8000 g;

triethanolamine 0.8000 g;

glucose 3.0000 g;

water 995.3975 g;

wherein, the purity of silver of the nano-silver powder is ≧99.99%, particles of the nano-silver powder are 1-5 nm in size and are in spherical shape.

### EXAMPLE 4

The nano-silver composition for the treatment of lung cancer of the Examples 1 to 3 were prepared in a method including the following steps:

(1) Adding the carbomer into ½ to ⅔ of total amount of the water with mixing to obtain a mixture, which is then dispersed in a ultrasonic disperser for 2-5 min;

(2) adding the nano-silver powder and the triethanolamine into the mixture dispersed in step (1) to obtain a mixture, which is then dispersed in a ultrasonic disperser for 2-5 min;

(3) adding the glucose and remaining water into the mixture dispersed in step (2) to obtain a mixture, which is then dispersed in a ultrasonic disperser for 1-3 min;

(4) cooling the mixture dispersed in step (3) to 2-12° C. to obtain a mixture, which is dispersed in an ultrasonic atomizer into a mist, which is collected in a collecting device and is condensed to form an anticancer nano-silver composition;

wherein, the ultrasonic disperser used in step (1) is of band 2 frequency: 16-24 KHz; the ultrasonic disperser used in step (2) is of band 5 frequency: 40-65 KHz; the ultrasonic disperser used in step (3) is of band 4 frequency: 30-40 KHz; the ultrasonic atomizer used in step (4) is of band 15 frequency: 120-180 KHz.

### EXAMPLE 5

Experiments were carried out to test the effects of the anticancer nano-silver composition on proliferation of human non-small cell lung cancer A549

### 1. Materials

The anticancer nano-silver composition prepared by using nano-sliver No1 in Example 4 is marked as Nano-Silver 1#. The anticancer nano-silver composition prepared by using nano-sliver No5 in Example 4 is marked as Nano-Silver 5#. The Nano-Slivers 1# and 5# are as used as the test groups.

### 2. Method

An X-Celligence Real-Time Cell Analysis System was used to generate real time records on the effects of the drug on proliferation of cancer cells within 72 hours after drug administration. Culture plates of E-Plate are used and in each well of which was placed with 5,000 of cells (initial value), 190 μL of culture medium, and 10 μL of sample solution. Different groups were established with different amounts of Nano-Silver 1# and Nano-Silver 5#, i.e., 50, 10 and 2 μL groups; also there established a solvent control group (0.1% DMSO) and a medium control group (Medium) without cells; and the effects of nano-silver on the A549 cell proliferation were observed.

### 3. Results

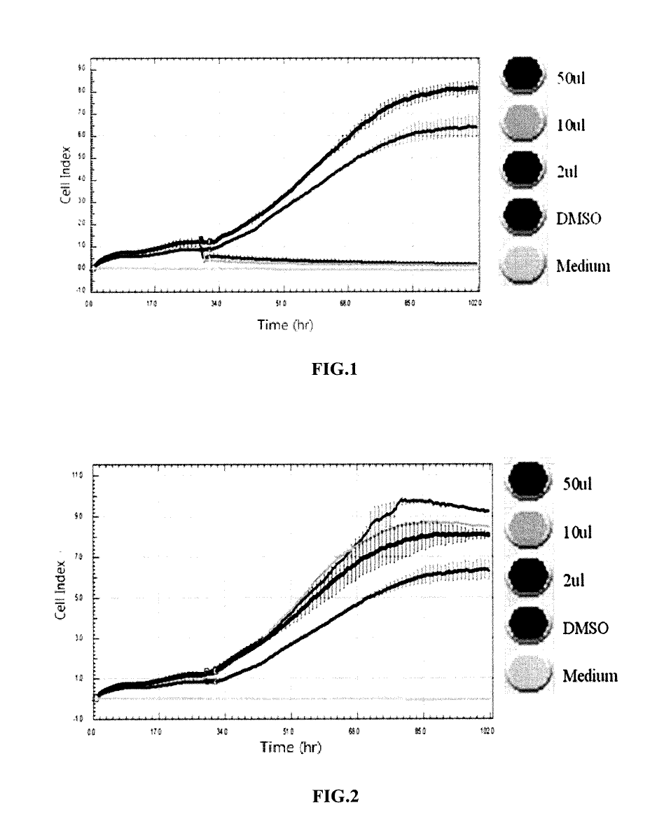

The effects of the Nano-Silver 5# on proliferation of A549 are illustrated in FIG. 1,and the effects of the Nano-Silver 1# on proliferation of A549 are illustrated in FIG. 2.

### 4. Conclusion

As illustrated in FIG. 2, the control group No.1 in various dosages (concentrations) showed no noticeable inhibition effects on proliferation of A549, which indicated that the control group No.1 does not inhibit the proliferation of A549; As illustrated in FIG. 1, the 50 μL (62.5 ppm) and 10 μL (12.5 ppm) groups of nano-silver No.5 completely inhibited on proliferation of A549, and caused the death of all of the cells; whereas the 2 μL (2.5 ppm) showed no noticeable inhibition effect on proliferation of A549. Thus, nano-silver No.5 inhibited proliferation of A549.

1. An anticancer nano-silver composition for treatment of lung cancer, the composition, on a basis of per kilogram of total weight, comprising:
nano-silver powder 12-200 mg;
carbomer 700-800 mg;
triethanolamine 700-800 mg;
glucose 2.8-3.2 g;
water as remaining;
wherein, purity of silver of the nano-silver powder is ≧99.99%, and particles of the nano-silver powder are 1-5 nm in size and spherical in shape;
wherein, the anticancer nano-silver composition is prepared by a method which comprises:
(1) preparing the nano-silver powder, carbomer, triethanolamine, glucose and water in a defined ratio;
(2) adding the carbomer into ½ to ⅔ of total amount of the water with mixing to obtain a mixture, which is then dispersed in a ultrasonic disperser for 2-5 min;
(3) adding the nano-silver powder and the triethanolamine into the mixture dispersed in step (2) to obtain a mixture, which is then dispersed in a ultrasonic disperser for 2-5 min;
(4) adding the glucose and remaining water into the mixture dispersed in step (3) to obtain a mixture, which is then dispersed in a ultrasonic disperser for 1-3 min;
(5) cooling the mixture dispersed in step (4) to 2-12° C. to obtain a mixture, which is dispersed in an ultrasonic atomizer into a mist, which is collected in a collecting device and is condensed to form an anticancer nano-silver composition.

2. The composition of claim 1, wherein the ultrasonic disperser used in step (2) is of band 2 frequency: 16-24 KHz.

3. The composition of claim 2, wherein the ultrasonic disperser used in step (3) is of band 5 frequency: 40-65 KHz.

4. The composition of claim 3, wherein the ultrasonic disperser used in step (4) is of band 4 frequency: 30-40 KHz.

5. The composition of claim 4, wherein the ultrasonic atomizer used in step (5) is of band 15 frequency: 120-180 KHz.

6. Use of the anticancer nano-silver composition of claim 1 for manufacturing a medicament to treat lung cancer.
